(19) United States
(12) Patent Application Publication
(10) Pub. No.: US 2025/0249062 A1
(43) Pub. Date: Aug. 7, 2025

(54) USE OF DIPSACI RADIX-DERIVED EXTRACELLULAR VESICLE-LIKE NANOPARTICLES IN PREPARATION OF DRUG FOR PREVENTING OR TREATING ORTHOPEDIC DISEASES

(71) Applicant: THE THIRD AFFILIATED HOSPITAL OF GUANGZHOU UNIVERSITY OF CHINESE MEDICINE, Guangzhou (CN)

(72) Inventors: Kewei ZHAO, Guangzhou (CN); Yue CAO, Guangzhou (CN); Qing ZHAO, Guangzhou (CN)

(21) Appl. No.: 19/077,789

(22) Filed: Mar. 12, 2025

Related U.S. Application Data
(63) Continuation of application No. PCT/CN2023/079368, filed on Mar. 2, 2023.

(30) Foreign Application Priority Data
Sep. 14, 2022 (CN) 202211117329.7

(51) Int. Cl. A61K 36/35; A61K 9/51; A61P 19/10

(52) U.S. Cl. CPC A61K 36/35; A61K 9/51; A61P 19/10; A61K 2236/31; A61K 2236/53

(57) ABSTRACT

Provided is a use of dipsaci radix-derived extracellular vesicle-like nanoparticles (DREVNs) in preparation of a drug for preventing or treating orthopedic diseases. In this application, EVs are creatively extracted from dipsaci radix and purified, and the physiological efficacy of the EVs is studied. It has been found that the EVs can be fully internalized by bone marrow mesenchymal stem cells (BMSCs), can promote the osteogenic differentiation of BMSCs by activating a BMP2/Smads signaling pathway, promote the calcified nodule formation in BMSCs, and promote the expression of osteogenic differentiation-associated genes ALP, OCN, RUNX2, and COL1, and have bone targetability in vivo. The EVs can be intragastrically administered to alleviate the osteoporosis (OP) in postmenopausal mice. Therefore, the EVs have the potential of being used to prepare drugs for preventing or treating orthopedic diseases, and provide a new strategy for the prevention or treatment of orthopedic diseases.

### CROSS-REFERENCE TO RELATED APPLICATION

This patent application is a national stage application of International Patent Application No: PCT/CN2023/079368, filed on Mar. 2, 2023, which claims the benefit and priority of Chinese Patent Application No. 2022111173297 filed with the China National Intellectual Property Administration on Sep. 14, 2022, the disclosure of which is incorporated by reference herein in its entirety as part of the present application.

### TECHNICAL FIELD

The present application belongs to the technical field of biomedicine, and relates to a pharmaceutical use of dipsaci radix-derived extracellular vesicle-like nanoparticles (DREVNs). Specifically, the present application relates to a use of DREVNs in preparation of a drug for preventing or treating orthopedic diseases.

### BACKGROUND

Osteoporosis (OP) is a systemic bone disease characterized by a small bone mass and the deterioration of a bone microstructure to cause increased bone fragility and susceptibility to fractures. OP predominantly affects the postmenopausal women and elderly men. One of the primary causes of the onset of OP is the imbalance between bone formation and bone resorption. Therefore, inhibiting the excessive activation of osteoclasts and enhancing the osteogenic differentiation are effective strategies for combating OP.

Dipsaci radix is the dried root of Dipsacus asper Wall. ex Henry from the Dipsacaceae family. Dipsaci radix has the effects of invigorating the liver and kidneys, strengthening the bones and muscles, and promoting the healing of fractures. Dipsaci radix is clinically used to treat conditions such as liver and kidney deficiency, traumatic injuries, injuries of tendons and muscles, fractures, etc. Extracellular vesicles (EVs) include a variety of proteins, lipids, and nucleic acids, and can play an important physiological role by mediating cell-to-cell communication. Almost all types of eukaryotic and prokaryotic cells secrete EVs. Plant EVs have a similar morphology to mammalian EVs, but there are few studies on the compositions, functions, etc. of plant EVs. Some studies have shown that plant EVs are components of the innate immune systems of plants and can exert antifungal effects. In addition, plant EVs have cross-species regulatory functions. Plant EVs can not only regulate the physiological functions of mammalian cells, but also intervene in and prevent the disease progression to play therapeutic roles for diseases. These research findings demonstrate that plant EVs, as a group of novel natural products, have the potential to become a promising candidate source for new drug development. Traditional Chinese medicinal materials are mostly plants, and have low costs and small side effects. However, there are limited studies on EVs derived from traditional Chinese medicinal materials. It remains unknown whether the extraction of EVs from dipsaci radix is feasible and whether the extracted DREVNs possess a biological activity.

Therefore, it is meaningful to develop a method for extracting EVs from dipsaci radix and conduct the in-depth research on functions of the extracted DREVNs, so as to explore an application potential of DREVNs in treating orthopedic diseases.

### SUMMARY

The present application provides a pharmaceutical use of DREVNs. Specifically, the present application relates to a use of DREVNs in preparation of a drug for preventing or treating orthopedic diseases.

In a first aspect, the present application provides a use of DREVNs in preparation of a drug for preventing or treating orthopedic diseases.

In the present application, EVs are creatively extracted from dipsaci radix and purified, and the physiological efficacy of the EVs is studied. It has been found that the EVs can be fully internalized by bone marrow mesenchymal stem cells (BMSCs), can promote the osteogenic differentiation of BMSCs by activating a BMP2/Smads signaling pathway, promote the calcified nodule formation in BMSCs, and promote the expression of osteogenic differentiation-associated genes (ALP, OCN, RUNX2, and COL1), and have bone targetability in vivo. Therefore, the EVs have the potential to be used to prepare drugs for preventing or treating orthopedic diseases, and provide a new strategy for the prevention or treatment of orthopedic diseases.

Preferably, the orthopedic diseases include any one selected from the group consisting of OP, a fracture, and osteoarthritis.

Preferably, a dosage form of the drug includes any one selected from the group consisting of a tablet, a capsule, a solution, an aerosol, a spray, an ointment, and a film,

Preferably, the drug further includes a pharmaceutically acceptable adjuvant, such as a diluent, a flavoring agent, a binder, a filler, a thickener, a lubricant, or a pH-regulating agent.

The DREVNs involved in the present application can be prepared by any method well known to those skilled in the art, and is preferably prepared by the following extraction method. The DREVNs prepared by this method have high purity, and exhibit an excellent effect of promoting the osteogenic differentiation of BMSCs and prominent bone targetability in vivo. The DREVNs have a particle size of 0 nm to 300 nm and are rich in nucleic acids, proteins, and lipids.

Preferably, the DREVNs are prepared by the extraction method including the following steps:
(1) juicing a dipsaci radix raw material, and filtering to produce a filtrate; and centrifuging the filtrate for removing impurities, and collecting a resulting supernatant; and
(2) subjecting the supernatant to ultra-high-speed centrifugation, collecting a resulting precipitate, and filtering the precipitate to obtain the DREVNs.

Preferably, the centrifuging in the step (1) includes at least three times of centrifugation.

Preferably, the centrifuging in step (1) includes first centrifugation, second centrifugation, and third centrifugation. The first centrifugation is conducted at a speed of 100 g to 500 g (such as 100 g, 150 g, 200 g, 250 g, 300 g, 350 g, 400 g, 450 g, or 500 g), the second centrifugation is conducted at a speed of 1,000 g to 5,000 g (such as 1,000 g, 1,200 g, 1,500 g, 1,700 g, 2,000 g, 2,200 g, 2,500 g, 2,700 g, 3,000 g, 3,500 g, 4,000 g, 4,500 g, or 5,000 g), and the third centrifugation is conducted at a speed of 8,000 g to 15,000 g (such as 8,000 g, 8,500 g, 9,000 g, 9,500 g, 10,000 g, 10,500 g, 11,000 g, 11,500 g, 12,000 g, or 15,000 g). The first centrifugation, the second centrifugation, and the third centrifugation each are independently conducted for 5 min to 40 min (such as 5 min, 10 min, 15 min, 20 min, 25 min, 30 min, 35 min, or 40 min).

Preferably, the ultra-high-speed centrifugation in step (2) is conducted at a speed of 100,000 g to 200,000 g (such as 100,000 g, 110,000 g, 120,000 g, 130,000 g, 140,000 g, 150,000 g, 160,000 g, 170,000 g, 180,000 g, 190,000 g, or 200,000 g) for 50 min to 100 min (such as 50 min, 55 min, 60 min, 65 min, 70 min, 75 min, 80 min, 85 min, 90 min, 95 min, or 100 min).

Other specific point values within the above numerical ranges can be selected and will not be repeated here.

Preferably, the filtering in step (2) is conducted with a 0.22 μm filter membrane.

In a second aspect, the present application provides a use of DREVNs in preparation of a bone-targeted formulation.

The present application has also creatively discovered that the DREVNs have bone targetability in animals. This discovery can be further applied to the preparation of bone-targeted formulations to allow the research on the pathogenesis of orthopedic diseases and the treatment of related diseases.

Preferably, the bone-targeted formulation further includes another drug for preventing or treating an orthopedic disease that is loaded in the DREVNs.

The another drug for preventing or treating an orthopedic disease is, for example, a calcium preparation, an estrogen drug, calcitonin, a bisphosphonate, or teriparatide.

In a third aspect, the present application provides a use of DREVNs in preparation of a promoter for osteogenic differentiation of BMSCs.

In a fourth aspect, the present application also provides a use of DREVNs in preparation of a promoter for osteogenic differentiation of BMSCs for a non-therapeutic purpose.

Since it has been discovered in the present application that the DREVNs have a significant effect of promoting the osteogenic differentiation of BMSCs, the DREVNs can be used as a promoter for osteogenic differentiation of BMSCs for a non-therapeutic and/or diagnostic purpose to conduct the theoretical scientific research on metabolic behaviors or others related to the differentiation of BMSCs.

In a fifth aspect, the present application also provides a method for promoting osteogenic differentiation of BMSCs, including: applying DREVNs to the BMSCs.

In a sixth aspect, the present application provides a use of DREVNs in preparation of a promoter for calcified nodule formation in BMSCs.

In a seventh aspect, the present application provides a use of DREVNs in preparation of a promoter for expression of alkaline phosphatase (ALP) and a promoter for expression of osteocalcin (OCN).

In an eighth aspect, the present application provides a use of DREVNs in preparation of a promoter for expression of ALP and a promoter for expression of OCN for a non-therapeutic purpose.

Since it has been discovered in the present application that the DREVNs have a significant effect of promoting the expression of ALP and OCN, the DREVNs can be used as a promoter for expression of ALP and a promoter for expression of OCN for a non-therapeutic and/or diagnostic purpose to conduct the theoretical scientific research on metabolic behaviors or others related to the differentiation of BMSCs.

In a ninth aspect, the present application also provides a method for promoting expression of ALP or OCN, including: applying DREVNs to the BMSCs.

In a tenth aspect, the present application provides a use of DREVNs in preparation of a BMP2/Smads signaling pathway agonist. Studies have shown that the DREVNs can significantly promote the expression of COL1, RUNX2, and BMP2 and promote the phosphorylation of Smad1/5/9.

Compared with the prior art, the present application has the following beneficial effects:

In the present application, EVs are creatively extracted from dipsaci radix and purified, and the physiological efficacy of the EVs is studied. It has been found that the EVs can be fully internalized by BMSCs, can promote the osteogenic differentiation of BMSCs by activating a BMP2/Smads signaling pathway, promote the calcified nodule formation in BMSCs, and promote the expression of osteogenic differentiation-associated genes (ALP, OCN, RUNX2, and COL1), and have bone targetability in vivo. Therefore, the EVs have the potential of being used to prepare drugs for preventing or treating orthopedic diseases, and provide a new strategy for the prevention or treatment of orthopedic diseases.

At present, there are no literature reports on the extraction method and function research for DREVNs. In the present application, DREVNs are extracted and studied in depth for the first time, which provides a new strategy for the research and development of plant-derived EVs and the research and treatment of orthopedic diseases.

### BRIEF DESCRIPTION OF THE DRAWINGS

FIG. 1 is a transmission electron microscopy image of DREVNs;

FIG. 2 shows the particle size distribution of DREVNs;

FIG. 3 shows the purity test results of DREVNs;

FIG. 4A-FIG. 4C show the detection results of DNA, RNA, proteins, and lipids in DREVNs by agarose gel electrophoresis, silver staining, and thin layer chromatography (TLC), where FIG. 4A shows the detection results of nucleic acids, FIG. 4B shows the detection results of proteins, and FIG. 4C shows the detection results of lipids;

FIG. 5 shows the morphologies of rat bone marrow mesenchymal stem cells (r-BMSCs);

FIG. 6 shows the flow cytometry identification results of third-generation r-BMSCs;

FIG. 7A shows the internalization conditions of DREVNs (1×10⁹ particles/mL and 5×10² particles/mL) by r-BMSCs at different time points that are determined by flow cytometry;

FIG. 7B shows the confocal microscopy images for internalization conditions of DREVNs (1×10⁹ particles/mL) by r-BMSCs at different time points;

FIG. 7C shows the confocal microscopy images for internalization conditions of DREVNs (5×10⁹ particles/mL) by r-BMSCs at different time points;

FIG. 8 shows the proliferation results of r-BMSCs under an action of DREVNs that are determined by a CCK-8 method;

FIG. 9A shows the statistical results of expression levels of ALP that are detected by enzyme-linked immunosorbent assay (ELISA);

FIG. 9B shows the statistical results of expression levels of OCN that are detected by ELISA;

FIG. 10A shows the images of alizarin red staining;

FIG. 10B shows the quantitative statistical results of abilities to promote the calcified nodule formation;

FIG. 11A-FIG. 11D show the expression levels of osteogenic differentiation-associated genes (ALP, OCN, RUNX2, and COL1) that are detected by quantitative real-time polymerase chain reaction (qRT-PCR), where FIG. 11A, FIG. 11B, FIG. 11C, and FIG. 11D show the relative expression levels of ALP, OCN, RUNX2, and COL1, respectively;

FIG. 12A-FIG. 12F show the expression results of osteogenic differentiation markers and key molecular proteins for a BMP2/Smads signaling pathway after a DREVN action that are detected by Western blot (WB), where FIG. 12A shows the expression of the osteogenic differentiation markers after the DREVN action that are detected by WB; FIG. 12B is a statistical chart for relative expression levels of COL1; FIG. 12C is a statistical chart for relative expression levels of RUNX2; FIG. 12D shows the expression of the key molecular proteins for the BMP2/Smads signaling pathway after the DREVN action that are detected by WB; FIG. 12E is a statistical chart of phosphorylation ratios of Smad1/5/9; and FIG. 12F is a statistical chart of relative expression levels of BMP2;

FIG. 13A shows the overall images and the images of organs (heart, liver, spleen, lungs, kidneys, artery, and femurs) for a bone-targeting test of DREVNs;

FIG. 13B is a statistical chart of fluorescence signal intensities of organs (heart, liver, spleen, lungs, kidneys, artery, and femurs) in a DiR group;

FIG. 13C is a statistical chart of fluorescence signal intensities of organs (heart, liver, spleen, lungs, kidneys, artery, and femurs) in a DiR-DREVN group;

FIG. 14A shows the flow cytometry results of fluorescence signal intensities in peripheral blood mononuclear cells;

FIG. 14B shows the flow cytometry results of fluorescence signal intensities in BMSCs;

FIG. 14C is a statistical chart of flow cytometry results of fluorescence signal intensities in peripheral blood mononuclear cells;

FIG. 14D is a statistical chart of flow cytometry results of fluorescence signal intensities in BMSCs;

FIG. 15 is a statistical chart of uterus indexes for groups;

FIG. 16 is a statistical chart of femur weight/length ratios for groups;

FIG. 17 shows the serum ALP expression levels in groups that are detected by ELISA;

FIG. 18A-FIG. 18B show the statistical results of serum calcium and phosphorus contents in groups;

FIG. 19A-FIG. 19J show the results of a test to alleviate OP by DREVNs in vivo, where FIG. 19A shows the images of a coronal section and cross section and the three-dimensional reconstruction image of a distal femur of a mouse in each group that are acquired by Micro-CT, and FIG. 19B to FIG. 19J show the statistical results of BMD, BV, BV/TV, BS/TV, Tb.N, BS/BV, Tb.Sp., Tb.pf., and SMI values of each group, respectively; and

FIG. 20A-FIG. 20B show the expression levels of key molecules for a BMP2/Smads signaling pathway in bone proteins that are detected by WB, where A shows the expression levels of BMP2 and RUNX2 that are detected by WB and B shows the phosphorylation levels of Smad1/5/9 that are detected by WB.

### DETAILED DESCRIPTION OF THE EMBODIMENTS

The technical solutions of the present application will be further described below through specific examples. Those skilled in the art should understand that these examples only help understand the present application and should not be regarded as specific limitations to the present application.

Dipsaci radix involved in the following examples was purchased from Bijie, Guizhou, and was identified as a dried root of Dipsacus asper Wall. ex Henry from the Dipsacaceae family by the Pharmacy of Traditional Medicine of the Third Affiliated Hospital of Guangzhou University of Chinese Medicine.

### EXAMPLE 1

### Extraction of DREVNs

(1) 500 g of fresh dipsaci radix was taken, thoroughly washed with sterile water, cut into small pieces, juiced, and filtered. A resulting filtrate was collected in a clean 50-mL centrifuge tube, and centrifuged at 300 g and 4° C. for 10 min to remove floating cells.
(2) The resulting supernatant was collected in a 50-mL centrifuge tube, and centrifuged at 2,000 g and 4° C. for 20 min to remove dead cells, detached vesicles, etc.
(3) The resulting supernatant was collected in a 50-mL centrifuge tube, and centrifuged at 10,000 g and 4° C. for 30 min to remove dead cells, detached vesicles, apoptotic bodies, etc.
(4) The resulting supernatant was collected in a centrifuge tube special for an ultracentrifuge, and centrifuged at 135,000 g and 4° C. for 70 min. The resulting supernatant was discarded. The resulting precipitate was resuspended with pre-cooled 1× phosphate-buffered saline (PBS) and filtered with a 0.22 μm disposable syringe filter. Finally, DREVNs were collected in a sterilized EP tube and stored in a −80° C. freezer for later use.

### EXAMPLE 2

### Characterization of DREVNs

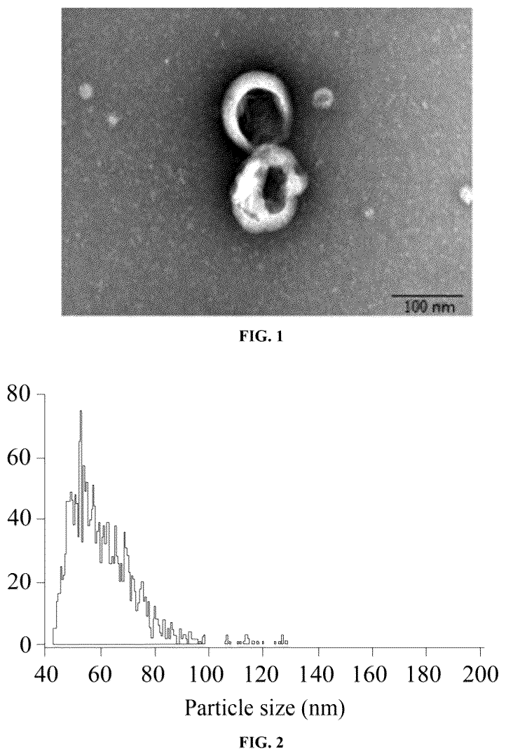

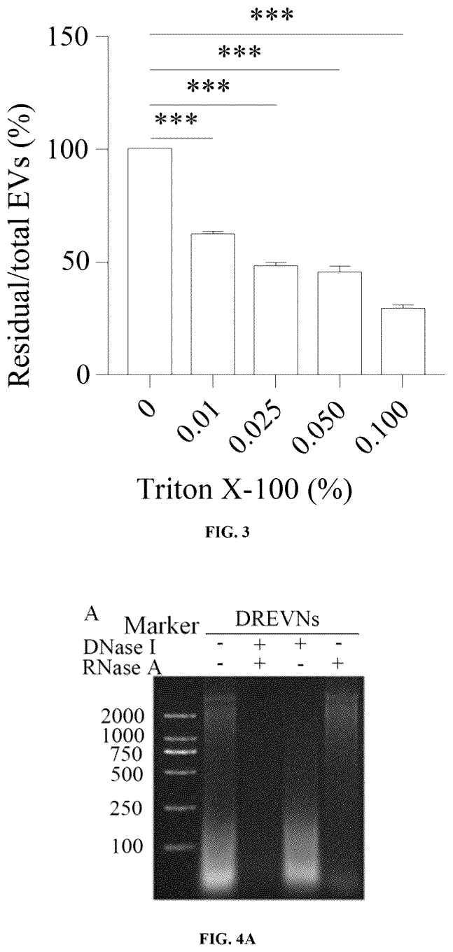

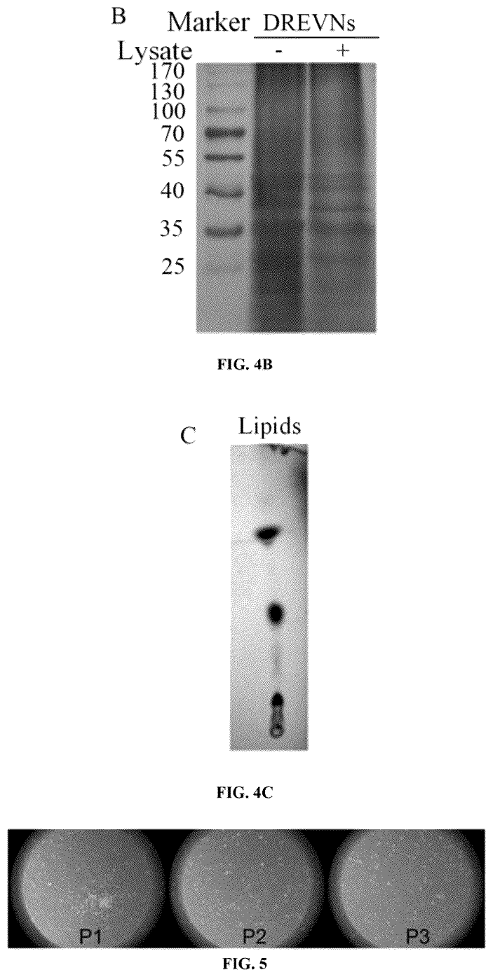

The DREVNs extracted in Example 1 were characterized for morphology, particle size, purity, and chemical composition.
(1) The morphology of DREVNs was observed under a transmission electron microscope. As shown in FIG. 1, the DREVNs had a concave surface, a typical cup saucer-shaped and disc-shaped vesicle structure, a clear and distinct cell membrane structure, a clear and sharp membrane boundary, a clean staining background, and an obvious contrast.
(2) particle size distribution of DREVNs was analyzed by a nanoparticle tracking analyzer. As shown in FIG. 2, the DREVNs had a particle size of 60.67±12.18 nm that was in the range of 0 nm to 300 nm and was in line with the EV size range reported in the literature, and were at a concentration of 1.27×10¹² particles/mL.
(3) The purity of DREVNs was detected by a polyethylene glycol octylphenyl ether (Triton X-100) permeabilization test. The DREVNs were subjected to membrane dissolution with Triton X-100 at different concentrations, and a particle number change was measured by nano-flow cytometry. As shown in FIG. 3, the purity of the extracted DREVNs was as high as 70%.
(4) DNA, RNA, proteins, and lipids in DREVNs were detected by agarose gel electrophoresis, silver staining, and TLC. Results are shown in FIG. 4A-FIG. 4C (where FIG. 4A showed the detection results of nucleic acids, FIG. 4B showed the detection results of proteins, and FIG. 4C showed the detection results of lipids). The results showed that DREVNs were similar to mammalian EVs, and were also rich in nucleic acids, proteins, and lipids.

The above results indicate that the DREVNs involved in the present application not only have a typical morphology, particle size, membrane structure, and chemical composition for EVs, but also have a high purity.

### EXAMPLE 3

### Internalization Test of DREVNs

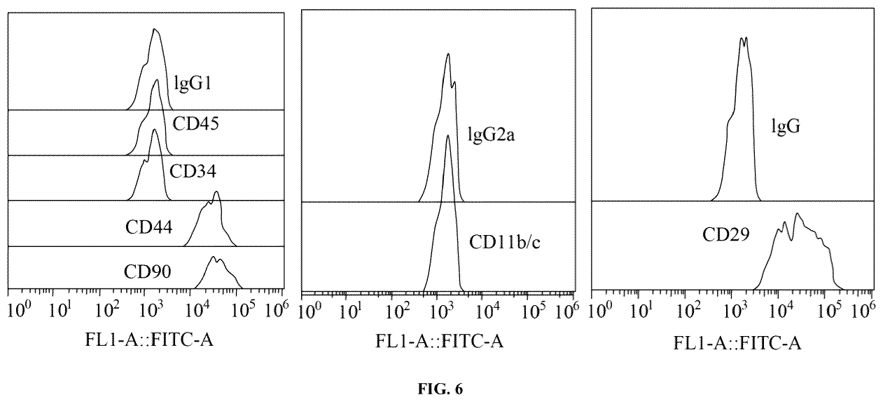

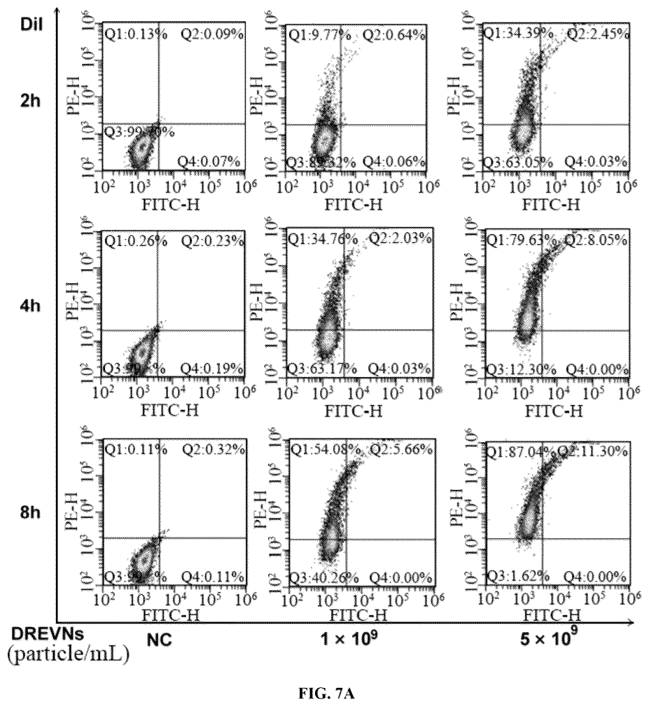

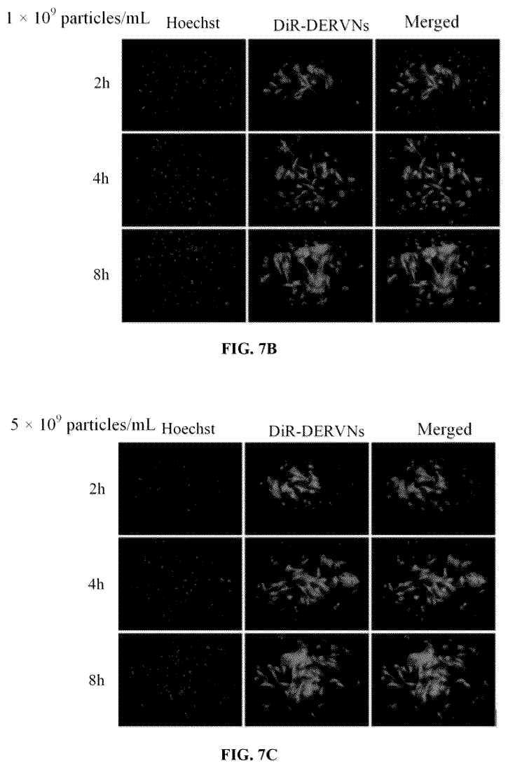

The full internalization of DREVNs by r-BMSCs was the prerequisite for subsequent action of DREVNs on r-BMSCs. Thus, whether DREVNs could be internalized by r-BMSCs must be verified before the follow-up experiment was conducted.
(1) Isolation and culture of r-BMSCs: A 4-week-old SD rat was taken, sacrificed through cervical dislocation, and soaked in 75% alcohol for 1 min. Femurs and tibias at both sides were collected under sterile conditions. Both ends of each bone were cut off, and a pre-cooled complete medium (89% L-DMEM+10% FBS+1% (penicillin+streptomycin)) was drawn with a 1-mL syringe to rinse the bone marrow until the bone turned white. The resulting cell suspension was collected and centrifuged at 1,200 rpm for 5 min. The resulting supernatant was discarded. The resulting precipitate was resuspended with a medium, inoculated in a culture flask, and cultured in a 37° C. and 5% CO₂ incubator, during which the medium was changed once every 3 d. When a cell confluency reached 80%, the digestion was conducted with a 0,25% trypsin solution, and the subculture was conducted at a ratio of 1:2. r-BMSCs of the third generation were taken for the subsequent experiment. The morphology of r-BMSCs is shown in FIG. 5.
(2) Identification of r-BMSCs: Specific steps: A. 100 μL of r-BMSC suspension (3×10⁶ cells/mL) was taken and added to an EP tube. B. 2 μL of primary antibody was added, thorough mixing was allowed, and incubation was conducted at 4° C. for 30 min. C. The resulting sample was washed twice with 200 μL of flow cytometry buffer each time and centrifuged at 250×g for 5 min, and the resulting supernatant was discarded. D. 100 μL of a flow cytometry buffer and 2 μL of a fluorescent secondary antibody were added, and cells were resuspended and incubated at 4° C. for 30 min. E. the resulting sample was washed twice with 200 μL of flow cytometry buffer each time and centrifuged at 250×g for 5 min, and the resulting supernatant was discarded. F. Cells were resuspended with 400 μL of flow cytometry buffer and then immediately loaded on a machine for testing. Results are shown in FIG. 6. r-BMSCs were positive for CD44, CD90, and CD29 and negative for CD34, CD45, and CD11b/c.
(3) Preparation of a Dil-DREVNs solution: 500 μL of DREVNs (1×10¹² particles/mL) and 500 μL of PBS were thoroughly mixed in a sterile EP tube, then 10 μL of DiR (DiI for flow cytometry) was added, thorough mixing was conducted, and the EP tube was incubated at 37° C. in the dark for 30 min and then centrifuged at 135,000×g and 4° C. for 70 min. The resulting supernatant was discarded. The resulting precipitate was resuspended with PBS, and subjected to supercentrifugation 3 times to remove the excess staining solution. After the last centrifugation, the resulting precipitate was resuspended with 500 μL of PBS to produce a DiR-DREVNs solution, which was stored in a −80° C. freezer for later use.
(4) Detection of internalization of DREVNs by r-BMSCs through flow cytometry and fluorescence microscopy: Labeled DREVNs at different concentrations (1×10⁹ particles/mL and 5×10⁹ particles/mL) were co-cultured with r-BMSCs for 2 h, 4 h, and 8 h. Then the internalization of DREVNs by r-BMSCs was detected by flow cytometry and fluorescence microscopy. Results are shown in FIG. 7A, FIG. 7B, and FIG. 7C (where FIG. 7A shows the internalization of DREVNs (1×10⁹ particles/mL and 5×10⁹ particles/mL) by r-BMSCs at different time points that was detected by flow cytometry, FIG. 7B shows the internalization of DREVNs (1×10⁹ particles/mL) by r-BMSCs at different time points that was acquired by confocal microscopy, and FIG. 7C shows the internalization of DREVNs (5×10⁹ particles/mL) by r-BMSCs at different time points that was acquired by confocal microscopy). DiI-DREVNs could be internalized by r-BMSCs, and an internalization degree increased with the increase of a Dil-DREVNs concentration and the extension of a co-incubation time. Most of the internalized Dil-DREVNs were located in the cytoplasm of r-BMSCs.

The above results showed that DREVNs could be fully internalized by r-BMSCs.

### EXAMPLE 4

### Research Test of an Effect and Mechanism of DREVNs to Promote the Osteogenic Differentiation

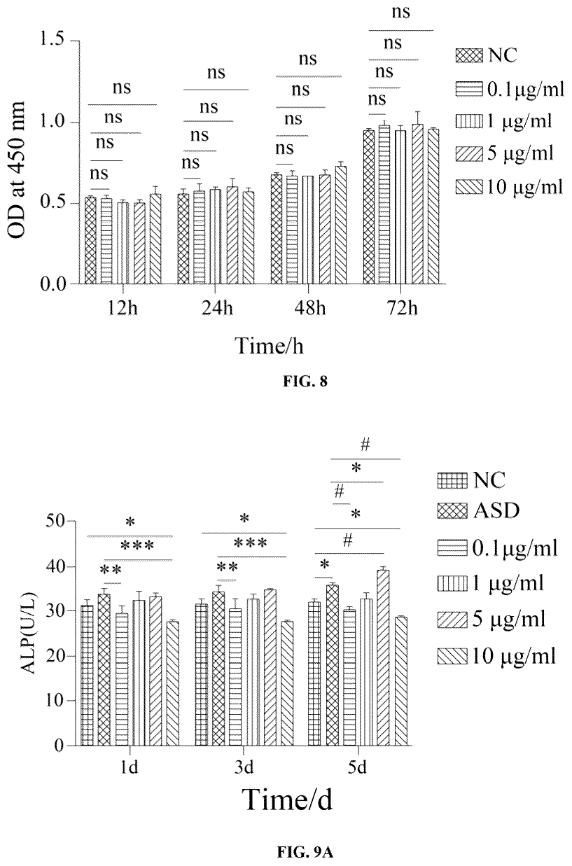

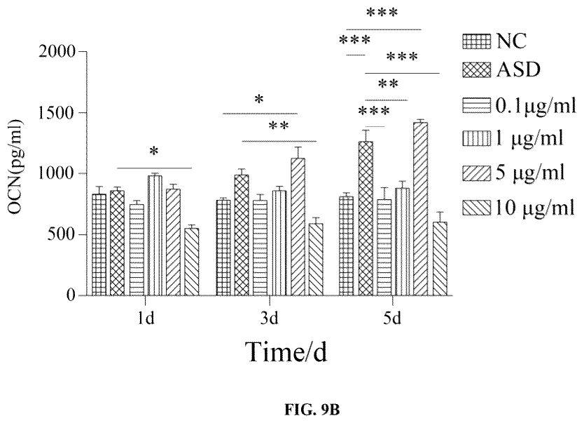

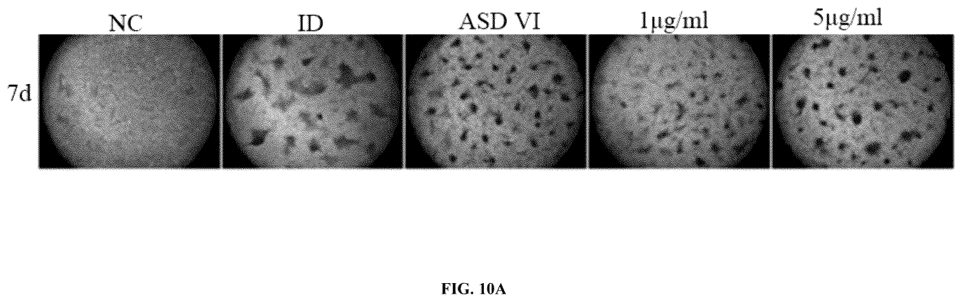

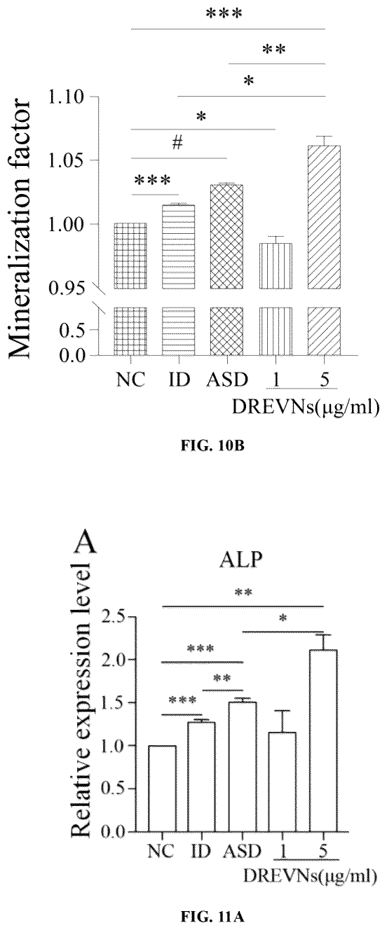

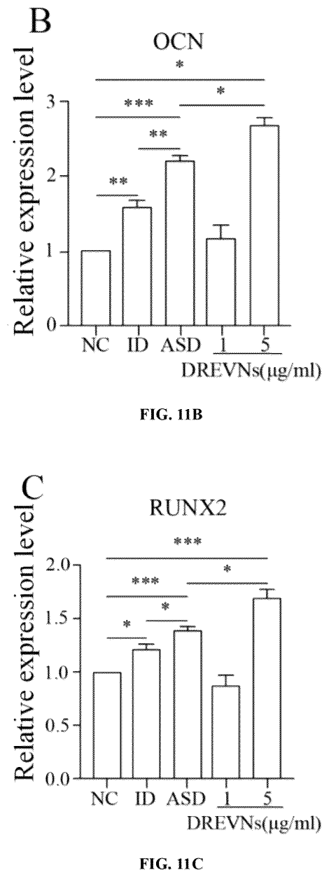

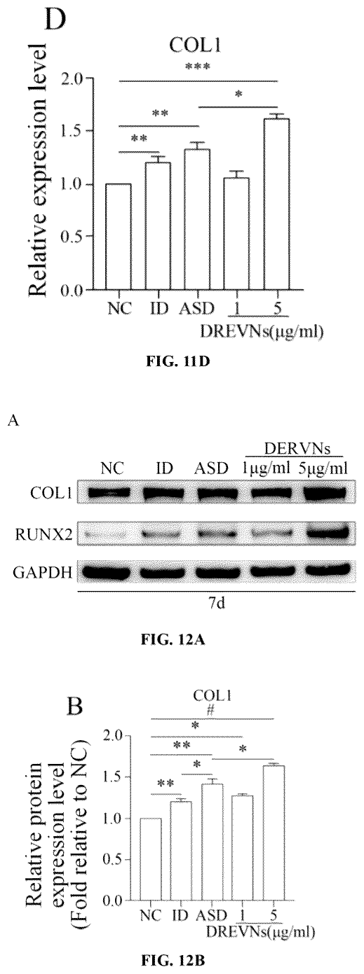

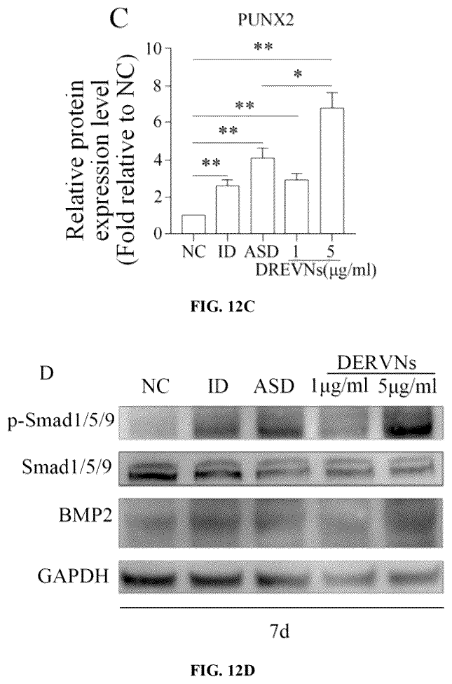

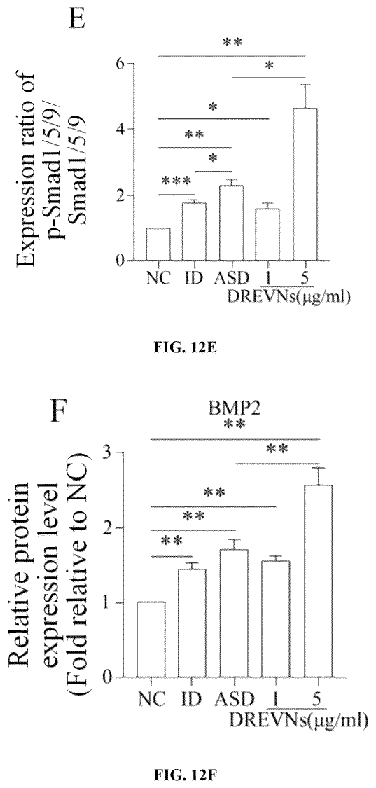

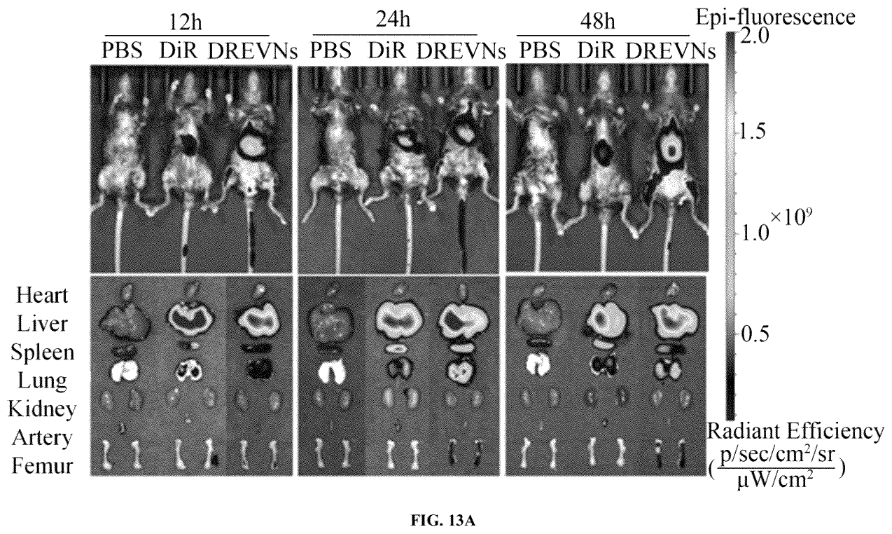

In order to verify a biological activity of DREVNs, a study at an in vitro cell level was first conducted in the present application to explore the effect and mechanism of DREVNs to promote the osteogenic differentiation of r-BMSCs.
(1) The protein concentration in DREVNs was detected by a BCA kit to be 8,000 μg/mL. In the subsequent study process, the consumption of DREVNs was measured with the protein concentration.
(2) Detection of influence of DREVNs on the proliferation of r-BMSCs by a CCK-8 method: r-BMSCs were inoculated in a 96-well plate at a cell density of 3×10³ cells/cm². DREVNs at different concentrations (0.1 μg/mL, 1 μg/mL, 5 μg/mL, and 10 μg/mL) were co-cultured with r-BMSCs. The absorbance of cells in each group at 450 nm was determined at 12 h, 24 h, 48 h, and 72 h. Results are shown in FIG. 8. DREVNs at the selected concentrations exhibited neither an inhibitory effect nor a proliferation-promoting effect for r-BMSCs.
(3) Detection of expression of ALP and OCN by ELISA: A cell culture method was the same as the method in (2). An ASD positive control group was added on the basis of the original CCK-8 grouping. The expression of ALP and OCN in a culture supernatant of each group was detected according to the instructions of ELISA kits for ALP and OCN. Results are shown in FIG. 9A and FIG. 9B (FIG. 9A shows the statistical results of ALP expression levels and FIG. 9B shows the statistical results of OCN expression levels). 1 μg/mL and 5 μg/mL DREVNs could promote the expression of ALP and OCN, and especially 5 μg/mL DREVNs exhibited the optimal expression-promoting effect. Therefore, the above two concentrations of DREVNs could be used as alternative concentrations for the subsequent cell experiments.
(4) Detection of the ability of DREVNs to promote the calcified nodule formation by alizarin red staining: r-BMSCs were inoculated in a 6-well plate at a density of 5×10⁴ cells/well, and divided into an NC group, an ID group (induction solution: 10 mmol/L of β-glycerophosphate sodium, 0.1 μmol/L of dexamethasone, and 50 mg/L of vitamin C), an ASD group (10⁻⁵ mol/mL of ASD), a 1 μg/mL DREVNs group, and a 5 μg/mL DREVNs group, with 3 wells for each group. After 7 days of induction, a medium was removed, and the cells were washed with PBS twice, fixed with 70% ethanol at 25° C. for 60 min, washed with PBS twice, stained with 0.1% alizarin red at 25° C. for 1 h, washed with PBS 3 times, and observed under a microscope. Results are shown in FIG. 10A and FIG. 10B (FIG. 10A shows the alizarin red staining results and FIG. 10B shows the quantitative statistical results of calcified nodule formation-promoting abilities). 5 μg/mL DREVNs had a stronger calcified nodule formation-promoting ability than the induction solution and ASD.
(5) Detection of influence of DREVNs on the osteogenic differentiation of r-BMSCs by qRT-PCR: The cell grouping and culture method were the same as those in (4). After 7 days of induction, the cells were collected in a sterilized EP tube with a TRIzol lysis buffer, and the EP tube was marked. The expression of the genes related to osteogenic differentiation (ALP, OCN, RUNX2, and COL1) was detected by qRT-PCR. Results are shown in FIG. 11A-FIG. 11D (FIG. 11A, FIG. 11B, FIG. 11C, and FIG. 11D show the relative expression levels of ALP, OCN, RUNX2, and COL1, respectively). 5 μg/mL DREVNs could significantly promote the expression of mRNAs for ALP, OCN, RUNX2, and COL1, and exhibited a better effect than the osteogenic induction solution and ASD.
(6) Detection of expression of osteogenic differentiation markers and key molecular proteins for a BMP2/Smads signaling pathway after a DREVN action by WB: The cell grouping and culture method were the same as those in (4). After 7 days of induction, the cells were lysed with a RIPA lysis buffer including a protease inhibitor and a phosphorylated protease inhibitor to extract proteins for follow-up experiments. Results are shown in FIG. 12A-FIG. 12F (FIG. 12A shows the expression of the osteogenic differentiation markers after a DREVN action that were detected by WB; FIG. 12B is a statistical chart for relative expression levels of COL1; FIG. 12C is a statistical chart for relative expression levels of RUNX2; FIG. 12D shows the expression of the key molecular proteins for the BMP2/Smads signaling pathway after the DREVN action that were detected by WB; FIG. 12E is a statistical chart of phosphorylation ratios of Smad1/5/9; and FIG. 12F is a statistical chart of relative expression levels of BMP2). 5 μg/mL DREVNs could significantly promote the expression of COL1, RUNX2, and BMP2 and promote the phosphorylation of Smad1/5/9.

The above results demonstrate that DREVNs can promote the osteogenic differentiation of r-BMSCs by activating the BMP2/Smads signaling pathway.

### EXAMPLE 5

### Bone Targeting Test for DREVNs

An in vivo targetability study was a prerequisite for the subsequent study of a biological activity of DREVNs in vivo. Therefore, targeted distribution of DREVNs in vivo was detected before an action effect and mechanism of DREVNs in vivo were verified.
(1) Preparation of DiR-DREVNs solution: The method was the same as the method in Example 3.
(2) In vivo imaging of animals:

Female C57BL/6J mice each were injected with 100 μL of the DiR-DREVNs solution through the tail vein. A blank control (100 μL of PBS was administered) and a positive control (100 μL of a DiR staining solution (DiR:PBS=1:50) was administered) were set. Fluorescence imaging was conducted at 6 h, 24 h, and 48 h after administration.

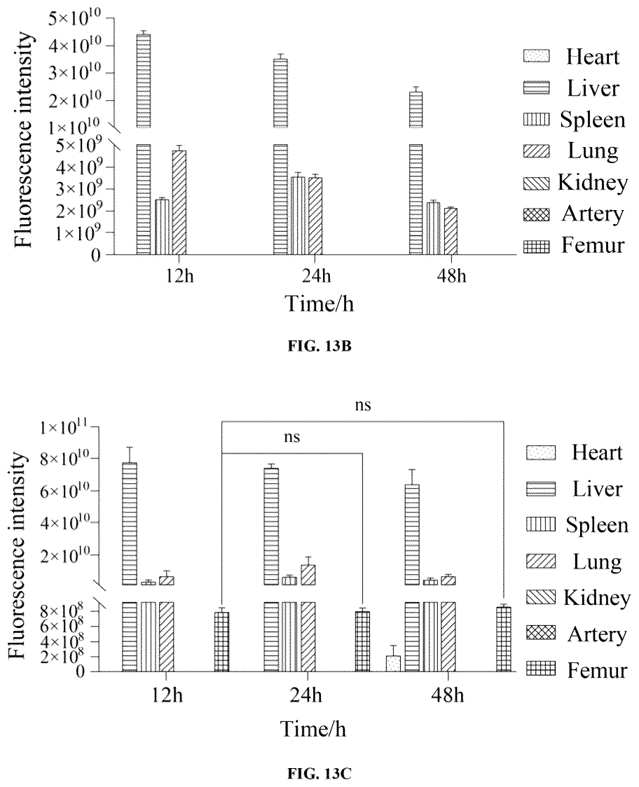

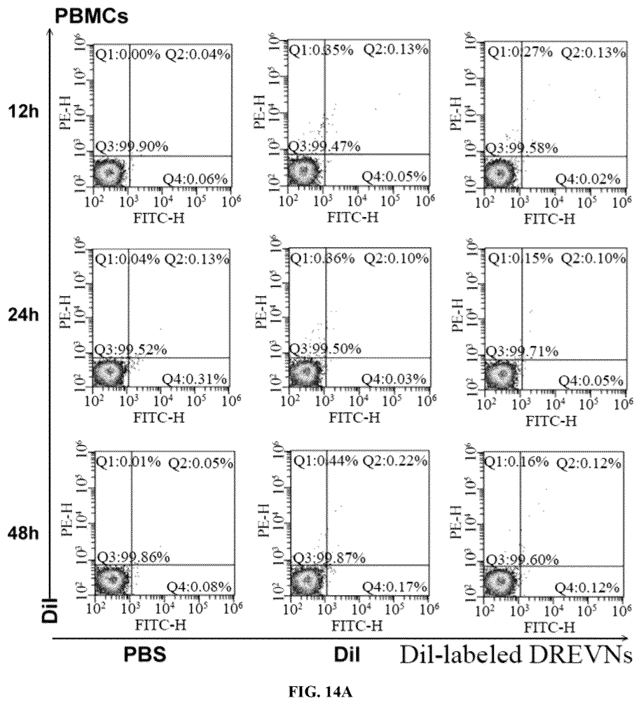

The overall imaging results are shown in FIG. 13A. No fluorescence signal was detected at the three time points in mice of the PBS group. A fluorescence signal occurred at the liver and tail of mice in the DiR group. A fluorescence signal occurred at the legs in mice of the DiR-DREVNs group in addition to the liver and tail, and the fluorescence signal in the legs tended to increase over time. Images of organs (heart, liver, spleen, lungs, kidneys, artery, and femurs) are shown in FIG. 13A, FIG. 13B, and FIG. 13C. No fluorescence signal was detected in organs of mice of the PBS group at any time point (FIG. 13A). In mice of the DiR group, fluorescence signals were mainly distributed in the liver, spleen, and lungs, but no fluorescence signal was detected in the heart, kidneys, artery, and femurs (FIG. 13A and FIG. 13B). In mice of the DiR-DREVNs group, in addition to the organs where fluorescence signals were distributed in the DiR group, a strong fluorescence signal was detected in the bone tissue, and tended to be intensified over time (FIG. 13A and FIG. 13C).
(3) Detection of targetability for peripheral blood mononuclear cells and BMSCs:

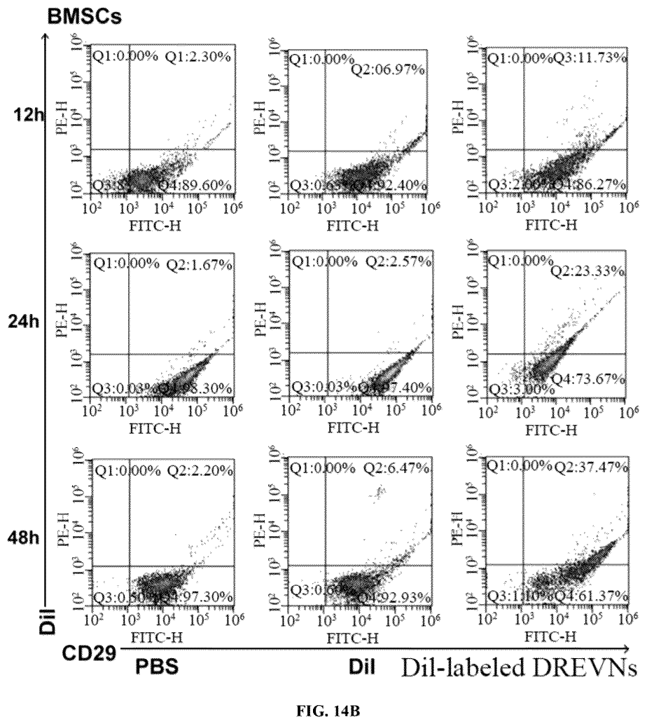

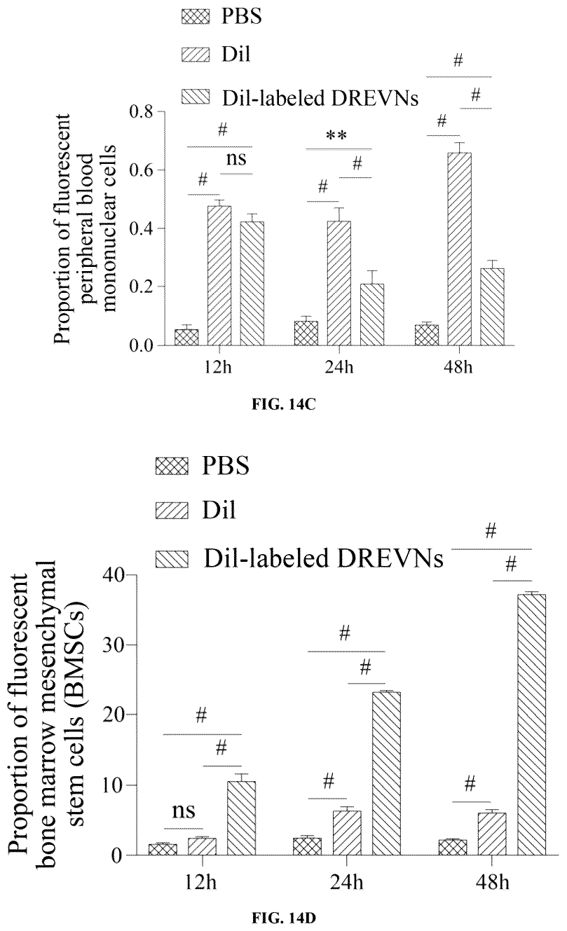

The selection of administration mode, grouping, and time point was the same as that in (2), but DiI was adopted as a fluorescent dye according to the requirements of the instrument. At 12 h, 24 h, and 48 h, eyeball removal, blood collection, and femur and tibia separation were conducted for mice in each group. Peripheral blood was placed in a sterile EP tube and diluted with an equal amount of normal saline for later use. BMSCs in the femur and tibia were rinsed and isolated with PBS, and the resulting cell suspension was collected for later use. The above cell suspension was slowly added to a corresponding 15-mL centrifuge tube with an equal volume of Ficoll separation solution, and centrifuged at 2,000 r/min and 20° C. for 25 min. A mononuclear cell layer was pipetted, placed in another 15-ml centrifuge tube, and centrifuged at 2,000 r/min and 20° C. for 15 min, and the resulting cell pellet was resuspended with 400 μL of PBS. Peripheral blood mononuclear cells could be detected directly by flow cytometry, and results are shown in FIG. 14A. BMSCs should be co-incubated with a CD29 primary antibody according to the procedures described in the identification operation for BMSCs, then a fluorescent secondary antibody was added, and the resulting sample was loaded on a machine for testing. Results are shown in FIG. 14B.

Statistical results are shown in FIG. 14C and FIG. 14D. DiR-DREVNs in mice of the DiR-DREVNs group did not exhibit targetability for peripheral blood mononuclear cells over time (FIG. 14A and FIG. 14C), while a proportion of BMSCs including DiR-DREVNs increased over time (FIG. 14B and FIG. 14D). The above results fully demonstrate that DREVNs have clear targetability for BMSCs, and BMSCs are a target for DREVNs to play a biologically-active role in vivo.

### EXAMPLE 6

### Establishment of Postmenopausal Osteoporosis (PMOP) Mouse Model

(1) Forty-eight 11-week-old female C57BL/6J mice were purchased from the Laboratory Animal Center in Guangzhou University of Chinese Medicine (animal license number: SYXK(Yue) 2018-0001).

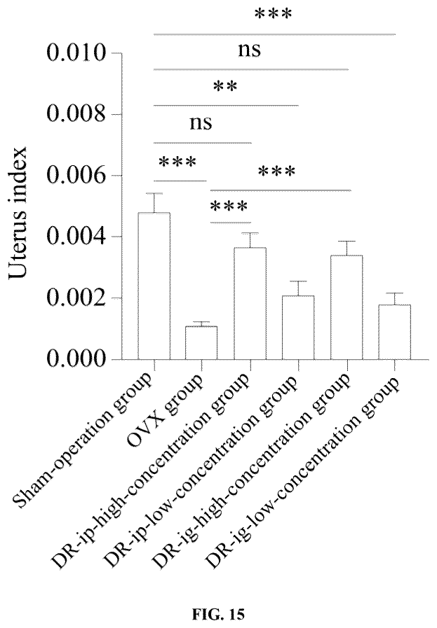

The mice were randomly divided into 6 groups: 1) a sham-operation group (n=8): mice were administered with 100 μL of normal saline; 2) an OVX group (model group) (n=8): mice were administered with 100 μL of normal saline; 3) a DREVNs-ip-high-concentration group (n=8): mice were intraperitoneally injected with 100 μL of high-concentration DREVNs (protein concentration: 800 μg/mL); 4) a DREVNs-ip-low-concentration group (n=8): mice were intraperitoneally injected with 100 μL of low-concentration DREVNs (protein concentration: 400 μg/mL); 5) a DREVNs-ig-high-concentration group (n=8): mice were intragastrically administered with 100 μL of high-concentration DREVNs (protein concentration: 1,600 μg/mL); and 6) a DREVNs-ig-low-concentration group (n=8): mice were intragastrically administered with 100 μL of high-concentration DREVNs (protein concentration: 800 μg/mL).
(2) The modeling method was as follows: Mice were intraperitoneally injected with pentobarbital (40 mg/kg) for anesthesia. Bilateral ovaries were removed from mice in the groups other than the sham-operation group through a dorsal incision to establish the PMOP model. In the sham-operation group, a specified amount of an adipose tissue around an ovary was removed through oophorectomy. On day 7 after the surgery, normal saline or DREVNs was administered continuously for 21 days (intragastric administration: 1 time/day, and intraperitoneal injection: 1 time/d). At the end of administration, a body weight of each mouse was measured. Eyeballs were removed, and blood was collected. The uterus was collected and weighed. Finally, the femurs were collected, measured for a length, and weighed.
(3) Uterus index refers to a ratio of uterus weight to body weight of a mouse. The uterus index can reflect the degree of uterine atrophy to reflect the ovarian resection. Thus, the uterus index is one of the important indexes to determine whether the PMOP modeling is successful. Results are shown in FIG. 15. The OVX group had a significantly-lower uterus index than the sham-operation group. This decline trend could be alleviated through the intraperitoneal or intragastric administration of DREVNs at different concentrations. Particularly, the high-concentration DREVNs led to a prominent effect.

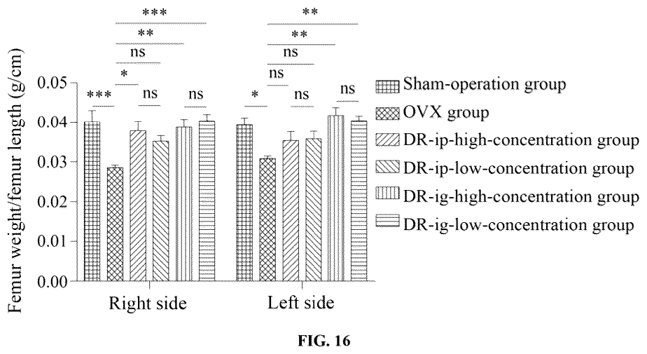

A ratio of the length to the weight of the femur could reflect a bone loss in a mouse. Results are shown in FIG. 16. Such ratios for left and right femurs of mice in the OVX group were significantly reduced. The increase of this ratio could be promoted through the intraperitoneal injection or intragastric administration of DREVNs at different concentrations, where the intragastric administration led to the optimal effect.

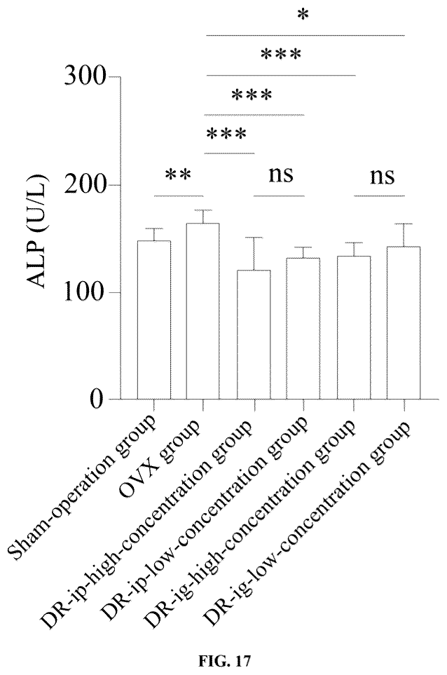

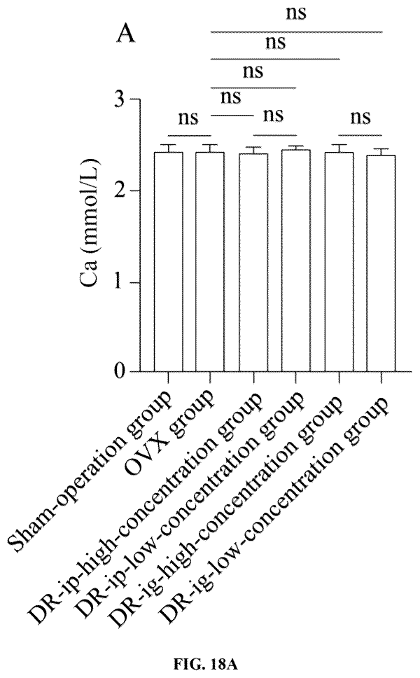

ALP is an important index for measuring the bone metabolism, and the expression level of serum ALP was detected by ELISA. Results are shown in FIG. 17. The expression of ALP in the OVX group was significantly increased. The intraperitoneal injection or intragastric administration of DREVNs at different concentrations all could reduce the expression of ALP.

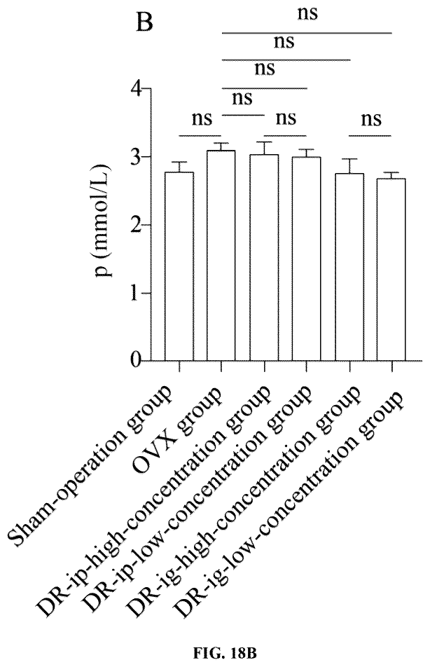

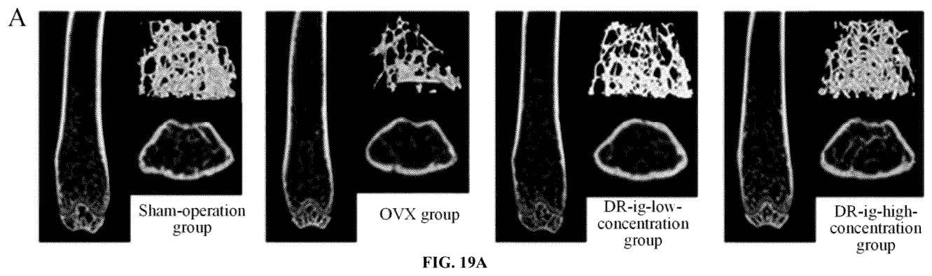

PMOP is primary OP. In the primary OP, serum calcium and phosphorus levels are generally within the normal ranges. Results are shown in FIG. 18A and FIG. 18B, serum calcium (FIG. 18A) and phosphorus (FIG. 18B) levels in mice of the OVX group both were within the normal ranges, which was in line with the characteristics of the PMOP model.

The above results showed that the PMOP model was successfully constructed and could be used for subsequent research.

### EXAMPLE 7

### Test of Alleviating OP by DREVNs In Vivo

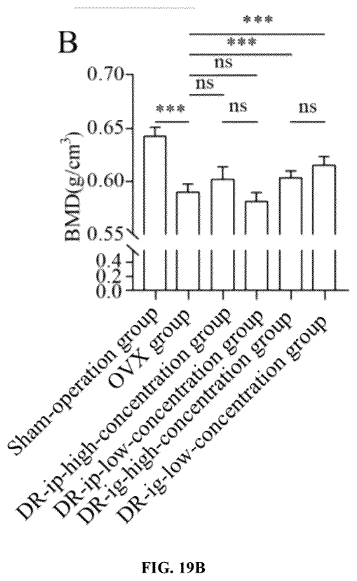

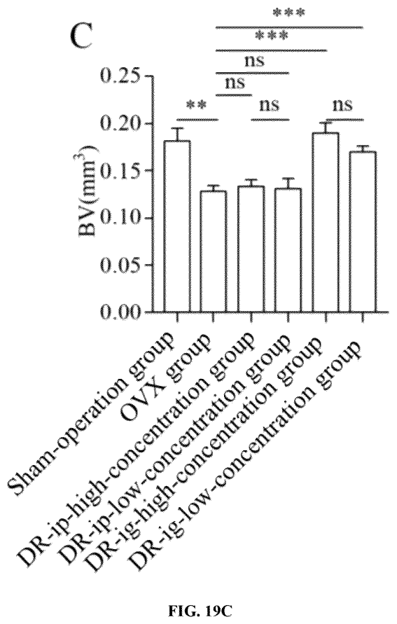

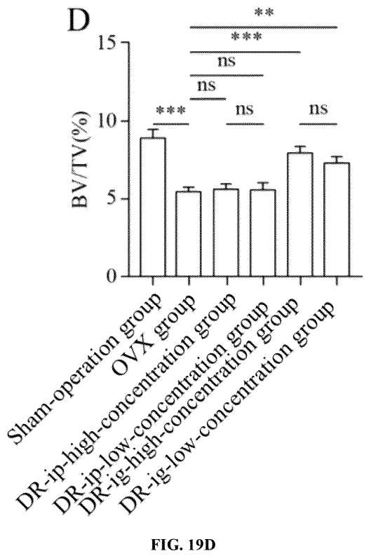

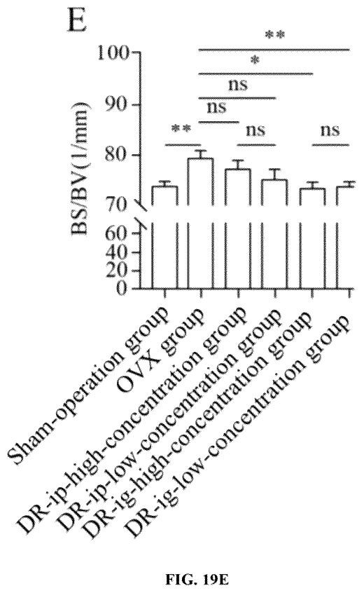

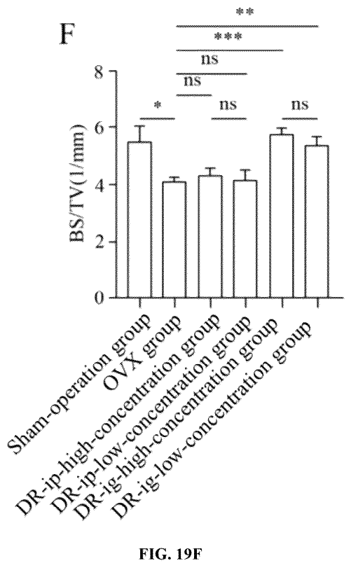

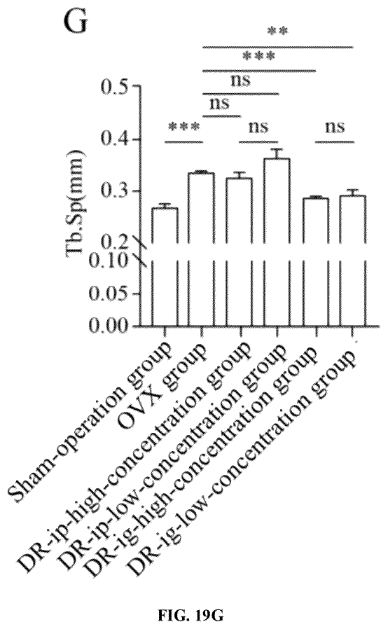

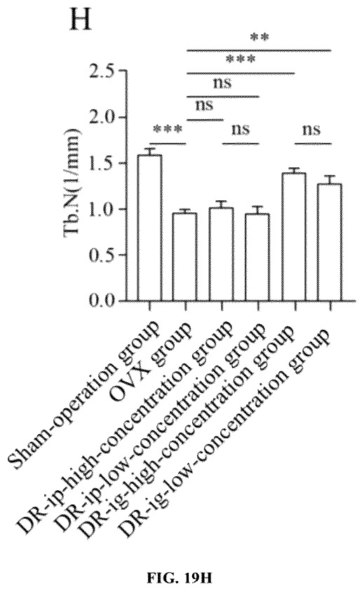

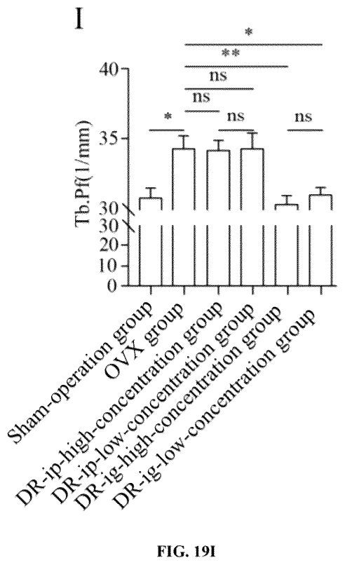

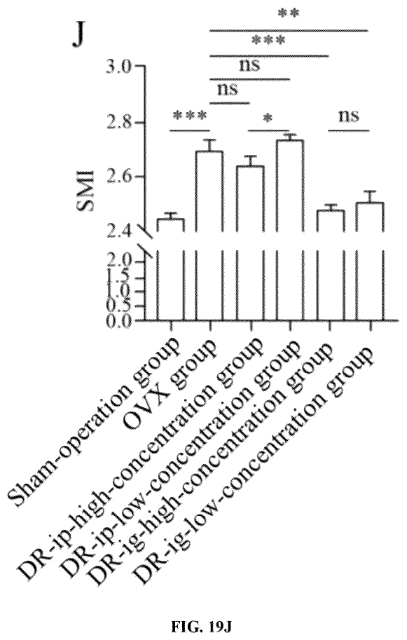

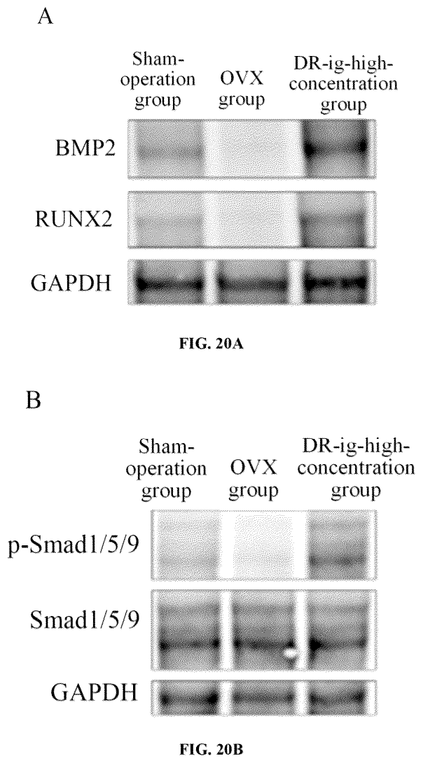

(1) Detection of bone tissue microstructures and bone parameters for distal femurs by Micro-CT: Images of a coronal section and cross section and a three-dimensional reconstruction image for the distal femur of a mouse in each group in Example 6 were acquired by Micro-CT (FIG. 19A). There was an obvious bone loss and bone microstructure damage in mice of the OVX group, and this tendency could be alleviated through the intragastric administration of DREVNs. The intragastric administration of DREVNs at different concentrations could reduce the BS/BV (FIG. 19E), Tb.Sp. (FIG. 19G), Tb.pf. (FIG. 19I), and SMI (FIG. 19J) while significantly increasing the BMD (FIG. 19B), BV (FIG. 19C), BV/TV (FIG. 19D), BS/TV (FIG. 19F), and Tb.N (FIG. 19H). There was no statistically significant difference between therapeutic effects of high-concentration and low-concentration DREVNs.
(2) The expression of key molecules for a BMP2/Smads signaling pathway in bone proteins was detected by WB. Detection results are shown in FIG. 20A-FIG. 20B (FIG. 20A shows the WB detection results of expression levels of BMP2 and RUNX2 and FIG. 20B shows WB detection results of phosphorylation levels of Smad1/5/9). It was found that, in the OVX group, the expression of BMP2 and RUNX2 decreased and the phosphorylation level of Smad1/5/9 decreased. The intragastric administration of DREVNs could promote the expression of BMP2 and RUNX2 and increase the phosphorylation level of Smad1/5/9, and there was no statistically significant difference between therapeutic effects of high-concentration and low-concentration DREVNs.

The above results indicate that the DREVNs involved in the present application have clear targetability for BMSCs and can alleviate OP by activating the BMP2/Smads signaling pathway in BMSCs.

The applicants declare that the use of the DREVNs of the present application in preparation of a drug for preventing or treating orthopedic diseases is described through the above examples in the present application, but the present application is not limited to the above examples, that is, the implementation of the present application does not have to rely on the above embodiments. Those skilled in the art should understand that any improvement to the present application, equivalent replacement of each raw material for the product of the present disclosure, addition of auxiliary ingredients, selection of specific methods, etc. all fall within the protection scope and disclosure scope of the present application.

Preferred embodiments of the present application are described in detail above, but the present application is not limited to specific details in the above embodiments. Various simple variations can be made to the technical solutions of the present application without departing from the technical ideas of the present application, and these simple variations fall within the protection scope of the present application.

In addition, it should be noted that various specific technical features described in the above specific embodiments can be combined in any suitable manner, provided that there is no contradiction. To avoid unnecessary repetition, various possible combination modes in the present application are not described separately.

What is claimed is:

1. A method for preventing or treating orthopedic diseases, comprising administering a medicament comprising dipsaci radix-derived extracellular vesicle-like nanoparticles (DREVNs) to a subject in need thereof.

2. The method according to claim 1, wherein the orthopedic diseases comprise any one selected from the group consisting of osteoporosis (OP), a fracture, and osteoarthritis.

3. The method according to claim 1, wherein a dosage form of the drug comprises any one selected from the group consisting of a tablet, a capsule, a solution, an aerosol, a spray, an ointment, and a film.

4. The method according to claim 1, wherein the drug further comprises a pharmaceutically acceptable adjuvant.

5. The method according to claim 1, wherein the DREVNs are prepared by an extraction method comprising the following steps:
(1) juicing a dipsaci radix raw material, and filtering to produce a filtrate; and centrifuging the filtrate for removing impurities, and collecting a resulting supernatant; and
(2) subjecting the supernatant to ultra-high-speed centrifugation, collecting a resulting precipitate, and filtering the precipitate to obtain the DREVNs.

6. The method according to claim 5, wherein the centrifuging in step (1) comprises at least three times of centrifugation:
preferably, the centrifuging in the step (1) comprises first centrifugation, second centrifugation, and third centrifugation, wherein the first centrifugation is conducted at a speed of 100 g to 500 g, the second centrifugation is conducted at a speed of 1,000 g to 5,000 g, and the third centrifugation is conducted at a speed of 8,000 g to 15,000 g; and the first centrifugation, the second centrifugation, and the third centrifugation each are independently conducted for 5 min to 40 min;
preferably, the ultra-high-speed centrifugation in the step (2) is conducted at a speed of 100,000 g to 200,000 g for 50 min to 100 min; and
preferably, the filtering in the step (2) is conducted with a 0.22-μm filter membrane.

7. A bone-targeted formulation comprising extracellular vesicle-like nanoparticles, wherein the extracellular vesicle-like nanoparticles are dipsaci radix-derived extracellular vesicle-like nanoparticles (DREVNs), and wherein the DREVNs are prepared by an extraction method comprising the following steps:
(1) juicing a dipsaci radix raw material, and filtering to produce a filtrate; and centrifuging the filtrate for removing impurities, and collecting a resulting supernatant; and
(2) subjecting the supernatant to ultra-high-speed centrifugation, collecting a resulting precipitate, and filtering the precipitate to obtain the DREVNs.

8. The bone-targeted formulation according to claim 7, wherein the bone-targeted formulation further comprises another drug for preventing or treating an orthopedic disease that is loaded in the DREVNs.

9. A method for promoting osteogenic differentiation of bone marrow mesenchymal stem cells (BMSCs), comprising applying the dipsaci radix-derived extracellular vesicle-like nanoparticles (DREVNs) to the BMSCs:
wherein the DREVNs are prepared by an extraction method comprising the following steps:
(1) juicing a dipsaci radix raw material, and filtering to produce a filtrate; and centrifuging the filtrate for removing impurities, and collecting a resulting supernatant; and
(2) subjecting the supernatant to ultra-high-speed centrifugation, collecting a resulting precipitate, and filtering the precipitate to obtain the DREVNs.

10. (canceled)

11. (canceled)

12. (canceled)
